(19) United States
(12) Patent Application Publication
(10) Pub. No.: US 2008/0140176 A1
(43) Pub. Date: Jun. 12, 2008

(54) Medical stent and devices for localized treatment of disease

(76) Inventors: Arthur A. Krause, Hollywood, CA (US); Walter K. Lim, Rancho Santa Fe, CA (US)

Correspondence Address: Dennis H. Lambert, 7000 View Park Drive, Burke, VA, 22015, US

(21) Appl. No.: 11/975,360

(22) Filed: Oct. 18, 2007

Related U.S. Application Data
(60) Provisional application No. 60/852,597, filed on Oct. 18, 2006.

(51) Int. Cl. A61F 2/84; A61F 2/82

(52) U.S. Cl. 623/1.11; 623/1.22; 623/1.15; 623/1.42; 623/1.31; 623/1.43

(57) ABSTRACT

A medical device for treatment of a stenosed body lumen includes an open-ended cylindrical body movable between a collapsed position and a radially expanded position pressed against the wall of the lumen is carried on a distal end of a catheter for insertion of the device into the lumen and placement at the stenosed site. In one embodiment the body sidewall is an open lattice-like structure, and a cover is attached to its outer surface. In another embodiment, concentric laminated tubes of dissimilar materials, such as, e.g., copper and silver, form the body. In a further embodiment stacked rings of different materials form the body. In a still further embodiment the device is temporarily placed in a body lumen for treatment of a stenosed site, after which the device is withdrawn. In all forms the body may have an outwardly flared inlet end to reduce turbulence.

This application claims the benefit of U.S. provisional patent application Ser. No. 60/852,597, filed Oct. 18, 2006.

### TECHNICAL FIELD

The present invention relates to medical devices. In particular, the present invention relates to stents for placement in a body lumen to correct or treat a diseased area in the lumen, as well as to devices for treatment of sites in a lumen that have been previously stented or previously unstented, and to devices for localized treatment of other diseased tissue.

### BACKGROUND ART

Diseased tissue generally is treated with surgical intervention, or drug therapy, or a combination of both. The therapeutic alternatives available for treatment of vascular disease, for example, which is caused by progressive blockage, or stenosis, of the blood vessels that perfuse the heart and other major organs, normally include surgical intervention to remove the blockage, i.e., replacement of the blocked segment with a new segment of artery, or the use of a catheter-mounted device such as a balloon catheter to dilate the artery. Both procedures are medical procedures whose purpose is to increase blood flow through an artery.

Inflation of a balloon to dilate the artery is known as angioplasty and is the predominant treatment for vessel stenosis. The increasing use of this procedure is attributable to its relatively high success rate and its minimal invasiveness compared with coronary bypass surgery. During angioplasty, a balloon catheter in a deflated state is inserted within a stenotic segment of a blood vessel and inflated and deflated one or more times to expand the vessel by compressing the built-up tissue or plaque in the vessel lumen to enlarge the opening and restore blood flow.

Angioplasty often permanently opens previously occluded blood vessels. However, a limitation associated with angioplasty is the abrupt closure of the vessel that may occur immediately after the procedure, and restenosis, which occurs gradually following the procedure and refers to the re-narrowing of an artery after an initially successful angioplasty. Additionally, restenosis is a chronic problem in patients who have undergone saphenous vein bypass grafting. Post-angioplasty closure of the vessel, both immediately after angioplasty (acute reocclusion) and in the long term (restenosis), is a major difficulty associated with angioplasty.

Because 30-50% of patients undergoing angioplasty will experience restenosis, the success of angioplasty alone is clearly limited as a therapeutic approach to coronary artery disease. Accordingly, stents of various configurations have been used to hold the lumen of a blood vessel open following angioplasty. Balloon angioplasty and associated implantation of a stent or stents compress the built-up tissue or plaque in a vessel lumen to enlarge the opening and restore blood flow. There is a multiplicity of different stents that may be utilized following angioplasty. Examples are disclosed in U.S. Pat. Nos. 5,766,710, 6,254,632, 6,379,382 and 6,613,084, and in published US applications 2002/0062147, 2003/0065346, 2003/0105512, 2003/0125800, 2003/0181973, 2003/0225450 and 2004/0127977. Most stents are compressible for insertion through small cavities, and are delivered to the desired implantation site percutaneously via a catheter or similar transluminal device. Once at the treatment site, the compressed stent is expanded to fit within or expand the lumen of the passageway. Stents are typically either self-expanding or are expanded by inflating a balloon that is positioned inside the compressed stent at the end of the catheter.

Stenting alone, however, may not always be successful because small muscle cell (SMC) proliferation and migration are intimately involved with the pathophysiological response to arterial injury. Thus, prevention of SMC proliferation and migration suggest the need for pharmacological intervention in the prevention of restenosis.

Accordingly, in addition to providing physical support to passageways, stents also are used to carry therapeutic substances for local delivery of the substances to the damaged vasculature. The therapeutic substances are typically either impregnated into the stent or carried in a polymer that coats the stent and are released from the stent or polymer once it has been implanted in the vessel. Substances that are commonly delivered from stents to inhibit development of restenosis and to reduce post-angioplasty proliferation of the vascular tissue, respectively, include: heparin and heparin fragments, colchicines, taxol, angiotensin converting enzyme (ACE) inhibitors, angiopeptin, and cyclosporin A. Numerous other agents are identified in U.S. Pat. No. 6,379,382, the disclosure of which is incorporated herein.

The local delivery of drug/drug combinations from a stent is advantageous because the scaffolding action of the stent prevents vessel recoil and closure, while the drug or drugs delivered from the stent prevent multiple components of neointimal hyperplasia or restenosis, and reduce inflammation and thrombosis. This local administration of drugs, agents or compounds to stented arteries may also have additional therapeutic benefit. For example, higher tissue concentrations of the drugs, agents or compounds may be achieved utilizing local delivery, rather than systemic administration.

In addition to maintaining higher tissue concentrations of a drug or drug combination, local delivery reduces systemic toxicity compared with systemic administration. Also, in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, and anti-thrombotic drugs, agents or compounds.

Notwithstanding the foregoing advantages, stents with rough surfaces exposed to blood flow can increase thrombosis. Local stagnation of blood and/or damage to red blood cells can occur due to interference to blood flow by the stent, and restenosis may still occur because tissue may grow through and around the lattice of the stent.

Further, coating of metal stents with a drug or beneficial agent generally requires the use of a polymer substrate to bond the agent to the stent, or stents with holes or depressions formed in them for storing the agent. Multiple drugs can be delivered by placing different drugs in different holes or depressions, or in different layers, but the holes or depressions tend to weaken the structure of the stent, and layering requires the drug carried by the underlying layer to pass through the top layer, or for the top layer to first dissolve or erode away.

When restenosis does occur at a previously stented site, conventional practice involves the implantation of a further stent at that site, but normally only one such additional stent can be implanted. After that, if restenosis occurs it is generally necessary to perform bypass surgery.

Accordingly, it would be advantageous to provide a stent having means for simultaneous delivery of multiple drugs or beneficial agents to a traumatized or diseased site in a vessel lumen while avoiding the problems associated with the prior art. It would also be advantageous to provide a stent having an auxiliary structure attached to the stent for delivering different pharmacologic agents and/or providing other benefits. Further, it would be advantageous to provide a stent constructed to avoid stagnation or pooling of blood at the stented site, and that did not cause trauma to blood flowing past the stent. Still further, it would be advantageous to provide means for localized treatment of vascular disease without the need for implanting a stent, or for “repair” of previously stented sites without the need for implanting a second stent at the previously stented site.

Other diseases, such as, for example, cancerous growths, tumors, and other localized diseases also are generally treated with surgical intervention, i.e., the surgical removal of the diseased tissue, and/or by systemic administration of drug or drug combinations. Systemic administration of drug or drug combinations to treat cancer usually requires the administration of large dosages of the drug or drugs in order to obtain an effective concentration of the drug or drugs at the diseased site. These high concentrations of the drugs are toxic to the patient, producing severe side effects that are not always tolerated well by the patient.

Accordingly, it would be advantageous to provide a means for the localized delivery of an appropriate concentration of a drug or drug combination to a diseased site, without the need for surgical intervention or the systemic administration of large dosages of a drug or drugs that are or can be toxic to the body.

### DISCLOSURE OF THE INVENTION

### A. Stents:

According to a first aspect of the present invention, an improved stent is provided for permanent implantation and delivery of a therapeutic agent or agents to a stenosed site in a body lumen. The stent includes an open-ended cylindrical body carried on a distal end of a catheter for insertion into the body lumen and placement at the stenosed site. The cylindrical body is movable between a collapsed position for insertion into the body lumen, and a radially expanded position pressed against the wall of the body lumen.

In one embodiment of the first aspect of the present invention, a stent body formed of interconnected struts or elements has an expandable auxiliary structure or cover attached to the stent and covering its outer surface when it is expanded, thus covering the openings or spaces between the struts and preventing extrusion or growth of tissue through the openings. In one form of this embodiment, the cover comprises a longitudinally pleated girdle. In another form, the cover comprises a coiled girdle. In a further form the cover comprises a plurality of overlapping plates attached to the stent body in a pattern similar to the scales of a fish. In a still further form the cover comprises bands abutting or closely spaced at their adjacent edges and wound in a spiral around the stent body from one end to the other. Each of the forms may be adapted in a manner as discussed in the following paragraphs to carry a drug or drugs on its outer surface. Further, the cover in each of these forms may be made of a variety of materials, including but not limited to copper, silver, foil, plastic, or a woven material. The cover in each form is welded to the stent body at appropriate points (e.g., one or both ends) to attach it to the stent prior to deployment of the stent and to retain it in place after deployment. The outer surface of the cover may be texturized to promote quicker growth of endothelial tissue. Texturizing the cover also facilitates adherence of polymer to the cover surface when drugs are applied to the cover in accordance with that technique. In the case of a pleated or coiled girdle, a single girdle may extend the full length of the stent body, or a plurality of girdles may be placed end-for-end on the stent body. In the fish scales version, the plates overlap one another when the stent is in its collapsed position, and as the stent is expanded they slide over each other to a slightly overlapping position or a non-overlapping position, depending upon how far the stent expands relative to its fully expanded position during manufacture. The plates are welded at only one end to the stent body, prior to moving the stent to its collapsed position, and the plates are not overlapping at that time. A lubricant may be placed on the plates and pleated or coiled cover in the various forms of the invention to facilitate sliding movement during expansion at the time of implantation. An advantage of the stent with a cover in accordance with the various forms of this embodiment of the invention may be that it will not be necessary to perform balloon angioplasty prior to stent implantation, since as the stent expands the surrounding cover acts similarly to the balloon in balloon angioplasty to press against the plaque and dilate the occluded or partially occluded lumen.

In another embodiment, the stent body is formed of spirally wound bands or ribbons interconnected at their opposite ends and slightly spaced apart at adjacent side edges. In a preferred construction the bands spiral through about 1.5 turns from one end of the stent to the other, but a different number of turns could be negotiated by the bands. This construction imparts a swirl motion to blood flowing through it, while at the same time presenting a relatively smooth interior surface to the blood flow, thereby helping to prevent formation of stagnant pools of blood without imposing turbulence or shear stresses on the blood.

In a further embodiment of the first aspect of the invention, different parts of the stent body are constructed of dissimilar metals and/or other materials selected for their different properties. In a preferred construction, the different materials are exposed at different portions of the stent body. In one form of this embodiment, the different materials are incorporated in different layers or laminations that are formed into concentric tubes and then cut with a laser or other suitable means to form the lattice structure of the stent, with one material exposed at the inner surface of the stent body and another material exposed at the outer surface. For example, an intermediate layer could comprise stainless steel, selected for its strength, an outer layer could comprise copper, selected for its therapeutic properties, and an inner layer could comprise another material selected for its particular properties. Copper ions, for example, break down or catalyze nitrosothiols in the blood to produce nitric oxide, which relaxes blood vessels, increases blood flow, and prevents clot-forming platelets from attaching to implant surfaces. In another form of this embodiment, the concentric tubes can be formed with segments or strips of different materials extending over only part of the circumference of the stent body, whereby not only can different materials be exposed at the inner and outer surfaces of the stent, but different materials can be exposed at different locations around its circumference. In a further form of this embodiment, rings of different materials are stacked and sonic or spot welded to each other to form a tubular structure, with different materials exposed along the length of the stent. Any number of rings can be employed, wherein succeeding rings along the length of the stent may comprise, for example, silver, steel, copper; silver, steel, copper; silver, steel, copper, and so on.

In a still further embodiment, the stent body is formed by a plurality of interconnected struts or elements forming a lattice structure having openings therethrough, and a plurality of enlarged pads or depots are provided at the intersections of at least some of the struts for carrying a therapeutic agent, or different therapeutic agents on different pads. The drug or drugs may be held in holes formed through the pads, or in depressions or a roughened surface formed in the surface of the pads, or in other ways known in the art, such as in a polymer coating on the pads, and the like. The various forms of this embodiment avoid the problems associated with prior art stents, wherein the drug or drugs are placed in openings or depressions formed in the stent structure itself, thus weakening the stent structure, or are carried either directly on the stent body or imbedded in a polymer substrate coated on the stent body and thus subject to dislodgement as the stent body expands during implantation.

The stent body in any or all of the forms of the invention may be coated with Teflon on at least its inner surface. One of the advantages of Teflon-coating of the stent is to ease blood flow through the stent channel. Additionally, adherence of blood platelets to the inner walls of the stent will be resisted. Coating of the stent body with Teflon is possible in the embodiments of the present invention because the girdles, plates, ribbons and pads attached to the outer surface of the stent body in the various embodiments carry the drug or drugs. Obviously, when the stent is coated with Teflon a drug or copolymer for carrying the drug cannot be adhered to the stent body, as in conventional stents.

In all of the preceding embodiments, and especially the swirl-inducing embodiment, the stent body may have a slightly outwardly flared inlet end. It has been noted in many studies that as the blood flows through the vascular tunnel and hits the opening or beginning of an implanted stent, the end of the stent may disturb the flow of blood and cause stagnation, shear stress, and/or turbulence at this point. It may also cause disturbance of the blood flow as it passes through the vascular channel downstream of the stent. The slightly outwardly flared inlet end of the stent in this embodiment effectively reduces or eliminates this disturbance and prevents stagnation, shear stress, and/or turbulence caused by the stent.

The ribbons, plates, pleated or coiled covers, laminated tubes, stacked rings, and stents themselves in the various embodiments described above can be made of materials such as copper, silver, steel, zinc, chrome, carbon, gold, brass, tantalum, titanium, platinum, sulfur compounds, and/or alloys or compositions thereof, and other materials that produce the desired results.

The auxiliary structures applied to the outside of the stent body in accordance with the invention may be made dissolvable, in the manner of dissolvable sutures, for timed release of pharmacological agents embedded in the auxiliary structure, or for other desired purposes. Thus, after the auxiliary structures and any pharmacological agent carried thereon have accomplished their purpose they are absorbed into or expelled by the system, with the stent body remaining in place to hold the lumen open. Of course, the stent body could also be made bioabsorbable so that it also is absorbed into or expelled by the system after it has accomplished its purpose.

Various therapeutic substances can be applied in any desired manner and combination to the auxiliary structures, i.e., the ribbons, plates, and pleated or coiled covers, that are attached to the outside of the stent body in accordance with the present invention, or to the laminated tubes, stacked rings, or bands forming the stent bodies. In one embodiment the agents are provided only in spaced areas so that the material of the underlying structure is exposed between the spaced areas. The exposed areas can thus provide or produce additional biological or pharmacological benefit. For example, if the underlying structure is made of copper or silver it can impede or prevent restenosis through the production of, e.g., copper ions that catalyze the breakdown of blood chemicals to produce nitric oxide, as discussed above. If copper ions are relied upon in this manner as a preventative for stenosis and restenosis, then it would not be necessary to put drugs or medications on the stent for this same purpose.

The therapeutic substances can comprise, for example, anticoagulants, antiplatelets, and cytostatic agents. Compounds such as Lecithin, Allicin (a raw garlic extract) and/or onion extracts, and HDL, are examples of naturally occurring substances that can be used. Other examples include those identified in U.S. Pat. No. 6,379,382, the disclosure of which is incorporated herein, and heparin and heparin fragments, colchicine, taxol, angiotensin converting enzyme (ACE) inhibitors, angiopeptin, and cyclosporin A. These substances are exemplary only, and are not intended to be limiting on the present invention.

### B. Vascular and Stent Repair:

According to another aspect of the present invention, vascular repair apparatus is provided to treat or “repair” a diseased site without the need for implanting a first or subsequent stent. That is, the apparatus according to this aspect of the invention can be used to treat stenosis without the need for implanting a stent, or it can be used to treat restenosis at a previously stented site, thus obviating the need for implanting a second stent at that site. The apparatus comprises a catheter with a device on its distal end for temporary placement at the diseased site and delivery for a limited time of a therapeutic agent or treatment that dissolves plaque or otherwise treats the diseased site as desired or necessary.

According to one embodiment of this aspect of the invention, a swab, brush, or sponge-like structure is carried on the distal end of a catheter for mechanically abrading the built-up plaque or other diseased tissue at the stenosed site, while a suitable treatment agent, drug, substance or compound carried by the device is released onto the plaque or other diseased tissue.

In another embodiment, high or low frequency sound is emitted by the device against the stenosed site to, for example, break up and liquefy plaque.

A further embodiment uses a form of light energy, such as a laser, or UV light or radiation, to destroy or vaporize diseased tissue.

Another embodiment uses thermal energy, e.g., a high temperature or a low temperature, to treat the stenosed site.

A still further embodiment uses hydraulic energy, wherein a high pressure spray or jet of fluid is directed against the diseased tissue. The spray may be steady, pulsating, and/or swirling. The fluid can comprise any suitable fluid, including blood plasma, or white blood cells from the patient, or saline solution, and the like, and can carry drugs.

Yet another embodiment directs oxygenated blood plasma, or other oxygenated fluid carrier, or just oxygen, against the diseased tissue to destroy it.

The foregoing systems preferably are constructed so that they permit blood to continue to flow while they are in place. They can be used alone or in combination with drug therapy. Thus, any of the devices can administer along with their underlying treatment modality one or more agents, drugs, substances, compounds or combinations thereof to obtain the desired pharmacological effect. Examples of some substances that may be employed are naturally occurring substances such as Lecithin, heparin, garlic and onion extracts, omega 3 (fish oil), ginger extract, medical nicotine, capsicum, and nitric oxide. Other substances can include aspirin and the various statin drugs, and/or a gel-like coating of a cholesterol-dissolving or blood clot dissolving agent.

When the device is used to deliver a treatment agent such as a drug, medication or other treatment substance to the affected site, it can be coated with a non-toxic dissolvable material, such as sugar for example, that will prevent dissolution or loss of the treatment agent during transit of the device to the affected site. The coating can be selected so that it will be dissolved just prior to or just after the device arrives at the site, whereby all of the treatment agent is available for application to the site.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is a perspective view of a first form of a first aspect of the invention, wherein a longitudinally pleated cover is attached to and covers the stent.

FIG. 2 is an enlarged end view of the device of FIG. 1, depicting the manner in which the pleated cover is attached to the stent, shown with the stent collapsed or crimped to its contracted condition after manufacture, and with the cover shown in an exaggerated, partially unfolded state.

FIG. 3 is a view in side elevation of the device of FIG. 1, showing the stent and cover in their contracted condition.

FIG. 4 is a perspective view of the device of FIG. 1, showing the stent and cover in their expanded condition.

FIG. 5 is an end view of the device of FIG. 4.

FIG. 6 is a perspective view of a second form of the first aspect of the invention, wherein a coiled cover is attached to and covers the stent.

FIG. 7 is an enlarged end view of the device of FIG. 6, depicting the manner in which the coiled cover is attached to the stent, shown with the stent collapsed or crimped to its contracted condition after manufacture.

FIG. 8 is a view in side elevation of the device of FIG. 6, showing the stent and cover in their contracted condition.

FIG. 9 is a perspective view of the device of FIG. 6, showing the stent and cover in their expanded condition.

FIG. 10 is a side view in elevation of a third form of the first embodiment of the invention, similar to that shown in FIG. 9, but wherein plural coiled covers are arranged end-to-end along the length of the stent.

FIG. 11 is an end view of the device of FIG. 9 or 10.

FIG. 12 is a side view in elevation of a fourth form of the first embodiment of the invention, wherein circumferentially overlapped plates are attached to the stent body, similar to fish scales, with the device shown in its collapsed condition.

FIG. 13 is a plan or developed view showing how the plates of FIG. 12 are overlapped.

FIG. 14 is a plan or developed view showing how the plates of FIG. 12 are related to one another when the stent is in its expanded condition.

FIG. 15 is a plan view of one of the plates that can be used in the form of the invention shown in FIG. 12, wherein the plate has a plurality of holes or depressions formed in it for attaching a drug or drugs to the plate.

FIG. 16 is a side view in elevation of a fifth form of the first aspect of the invention, wherein the stent comprises a plurality of slightly spaced apart spirally wound bands extending along the length of the stent to impart a swirling motion to blood flowing through the stent.

FIG. 17 is a fragmentary view in side elevation showing how the inlet end of the stent according to any of the foregoing forms of the invention can be outwardly flared to facilitate smooth flow of blood entering the stent.

FIG. 18 is a developed view of the stent of FIG. 16.

FIGS. 19-21 are developed views of variations of the stent shown in FIG. 16, wherein depressions or openings are formed in the bands to hold a drug or drugs.

FIG. 22 is an exploded view of rings of dissimilar materials that may be stacked together to form a tubular stent body.

FIG. 23 is a side view in elevation of a stent body formed of stacked rings of dissimilar materials so that different materials are exposed along different parts of the length of the stent.

FIG. 24 is a perspective view of a stent body formed of plural layers of strips of dissimilar materials so that different materials are exposed at the inner and outer surfaces of the stent and at different circumferential portions of the stent.

FIG. 25 is a view in side elevation of a stent body comprising interconnected strut elements forming a lattice-like stent structure, wherein the strut elements are comprised of different materials in different zones along the length of the stent.

FIG. 26A is an end view of a stent formed of three concentric layers of different materials, including an outer layer of copper, an intermediate layer of steel, and an inner layer of silver.

FIG. 26B is an end view of a stent formed of two concentric layers of different materials, including an outer layer of copper and an inner layer of steel.

FIG. 27 is a side view in elevation of a tubular stent body according to one of the forms of the invention shown in FIG. 26A or 26B, prior to being cut to form a lattice-like structure.

FIG. 28 is a fragmentary side sectional view of a stent body according to FIG. 26A.

FIG. 29 is a side view in elevation of a stent formed of interconnected strut elements and having enlarged pads at some of the intersections for carrying a drug or drugs.

FIG. 30 is a perspective sectional view showing a stent in place in an artery.

FIGS. 31 and 32 are developed views of a stent such as that shown in FIG. 29, with the pads in FIG. 31 not having any depressions or openings therein, and the pads in FIG. 32 having depressions or openings formed therein for holding a drug or drugs.

FIG. 33 is a perspective view of a catheter and associated “repair” device in accordance with a second aspect of the present invention.

FIG. 34 is a fragmentary view in side elevation of a distal end portion of a catheter with a first form of “repair” device according to the second aspect of the invention attached thereto, wherein the first form of “repair” device comprises a sponge.

FIG. 35 is a fragmentary view in side elevation of a distal end portion of a catheter with a second form of “repair” device according to the second aspect of the invention attached thereto, wherein the second form of “repair” device comprises a balloon.

FIG. 36 is a fragmentary view in side elevation of a distal end portion of a catheter with a third form of “repair” device according to the second aspect of the invention attached thereto, wherein the third form of “repair” device comprises means for directing a spray or jets of fluid against a stenosed site.

FIG. 37 is a fragmentary view in side elevation of a distal end portion of a catheter with a fourth form of “repair” device according to the second aspect of the invention attached thereto, wherein the fourth form of “repair” device comprises means for directing ultrasound against a stenosed site.

FIG. 38 is a perspective end view of a “repair” device according to any of the immediately preceding forms of the invention, depicting how the device is hollow to permit blood to continue to flow while the device is in place at a stenosed site.

FIG. 39 is a longitudinal sectional view showing one of the “repair” devices in place at a stenosed site in an artery.

### BEST MODES FOR CARRYING OUT THE INVENTION

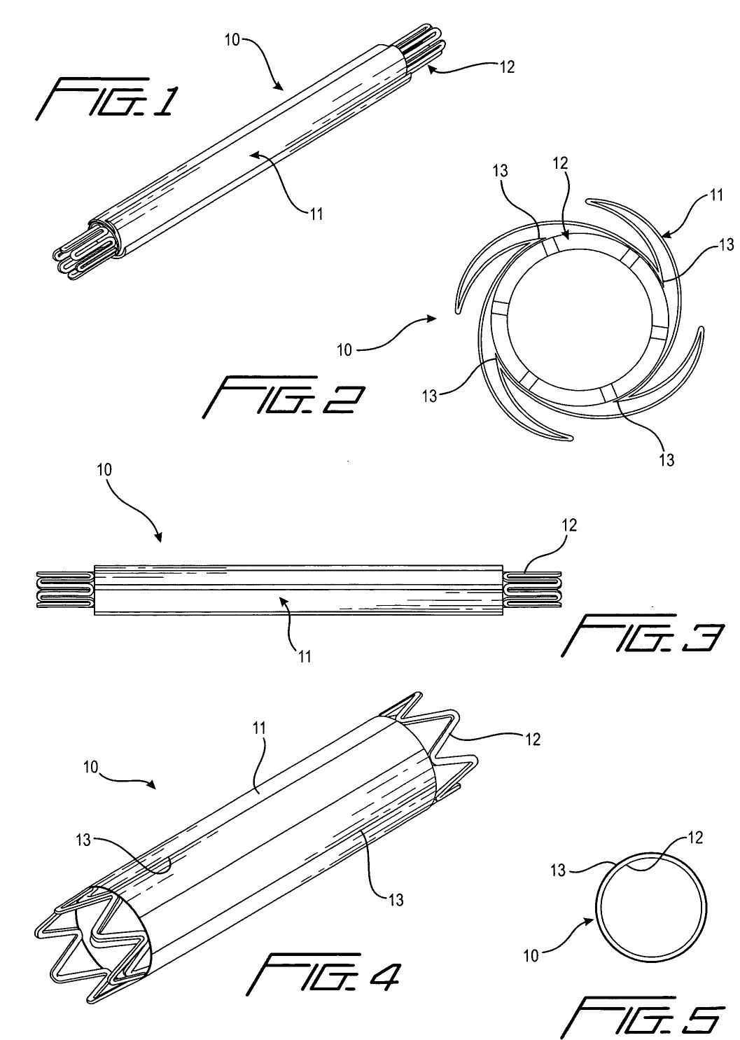

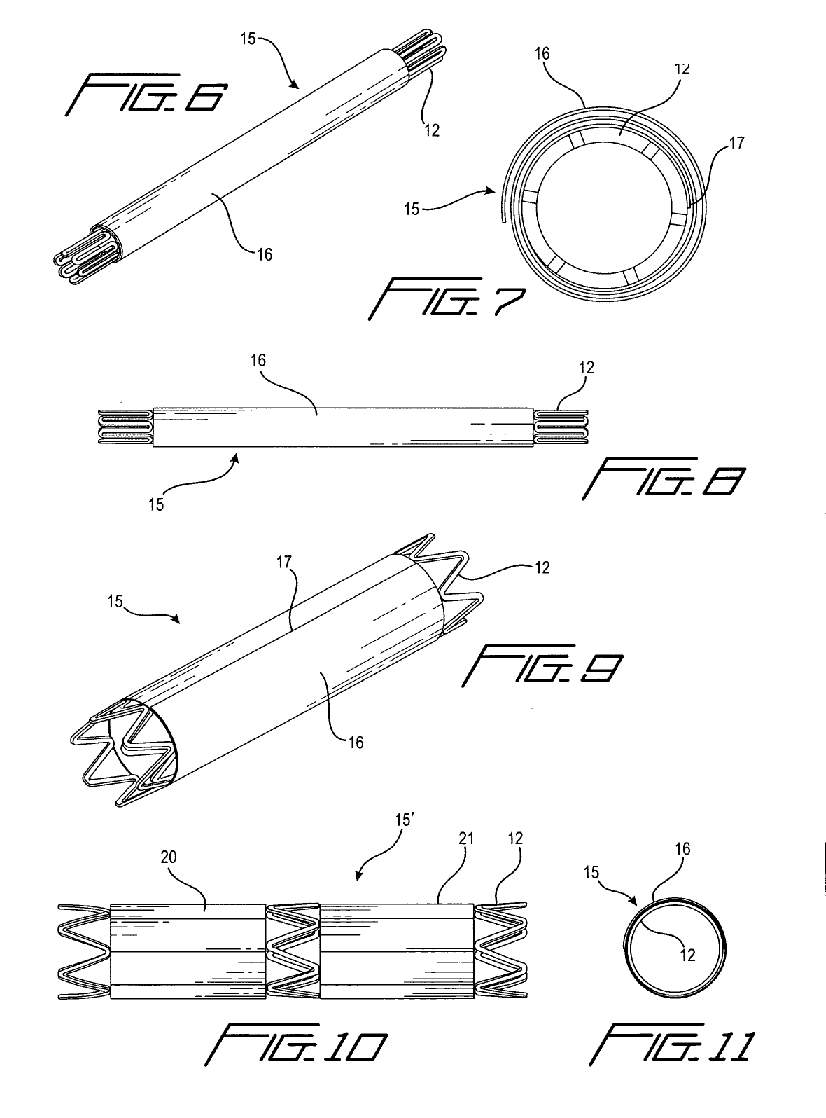

A first embodiment of a stent with auxiliary structure according to a first aspect of the invention is shown generally at 10 in FIGS. 1-5. In this form, a longitudinally pleated cover 11 is attached to a stent body 12, which may be of any suitable conventional construction, at longitudinally extending points 13 (see FIG. 2). The cover is applied to the stent body while the stent is in its as-manufactured expanded condition (see FIG. 4), and is attached by welding or other suitable fastening means. The stent and cover are then collapsed to a contracted condition as shown in FIGS. 1-3.

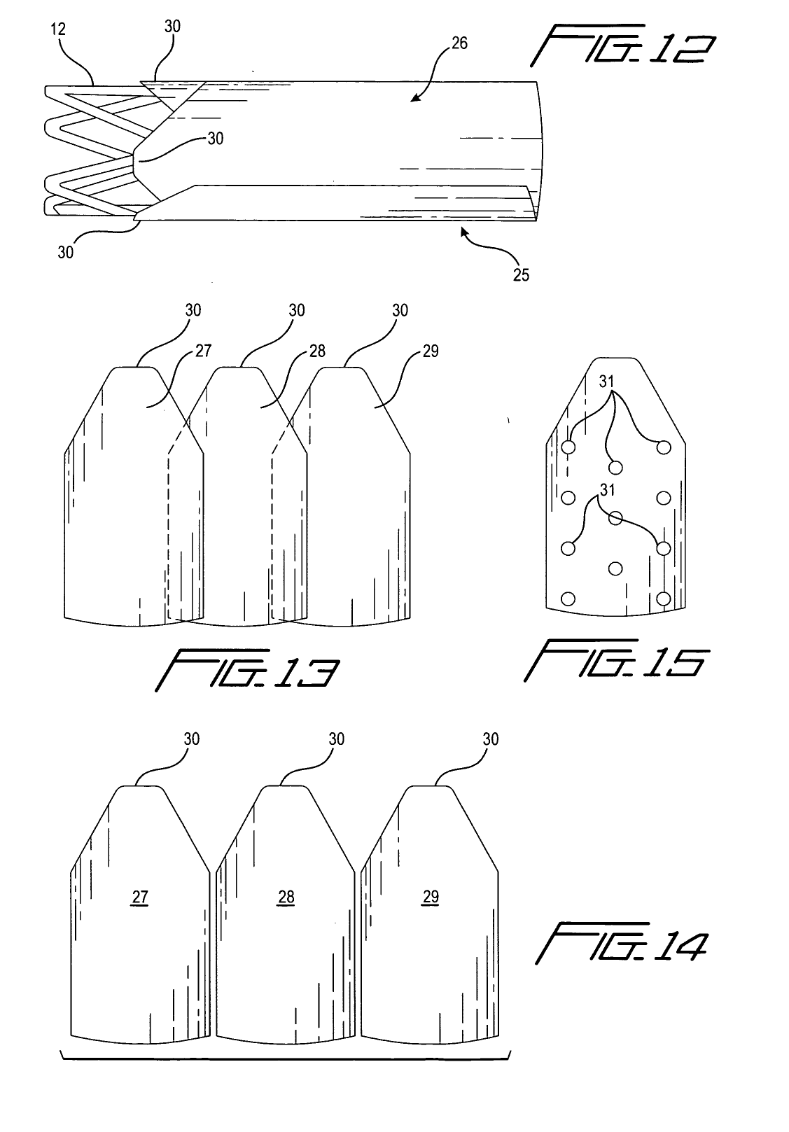

A second embodiment of a stent with auxiliary structure according to the first aspect of the invention is shown generally at 15 in FIGS. 6-9 and 11. In this form, a coiled cover 16 is attached to the stent body 12 along one edge 17 extending longitudinally of the stent. The coiled cover is applied while the stent is in its as-manufactured expanded condition as shown in FIGS. 9 and 11, after which the stent is collapsed and the cover coiled around it as shown in FIGS. 6-8.

FIG. 10 depicts a variation 15′ of the forms of invention shown in FIGS. 1-9 and 11, in that a plurality of covers 20 and 21 are applied to the stent body in end-to-end relationship along the length of the stent. The covers may be longitudinally pleated as in FIG. 1, or coiled as in FIG. 6.

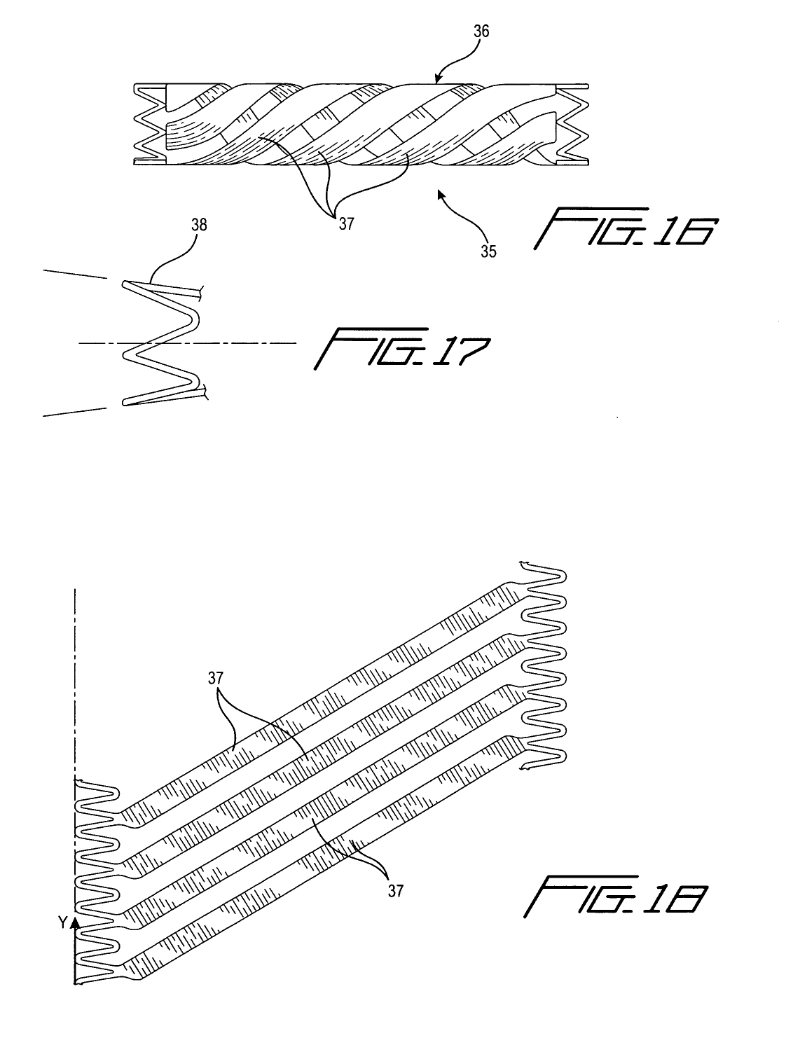

A third embodiment of the first aspect of the invention is indicated generally at 25 in FIGS. 12-14. In this embodiment, the cover 26 comprises a plurality of overlapping plates 27, 28, 29, fixed by any suitable means, such as by welding, at an upstream end 30 to the stent body 12 and left unattached over the rest of their length. The plates are attached to the stent body while the stent is in its expanded, as-manufactured condition, at which time the plates 27, 28, 29 preferably will not be overlapping, as depicted in FIG. 14. After the plates are attached, the stent and cover are collapsed to their contracted condition as depicted in FIGS. 12 and 13. The plates may be suitably treated, as by texturizing their surface (not shown), or providing depressions or holes 31 therein (FIG. 15), or providing a polymer coating, to hold a drug or drugs applied to the plates.

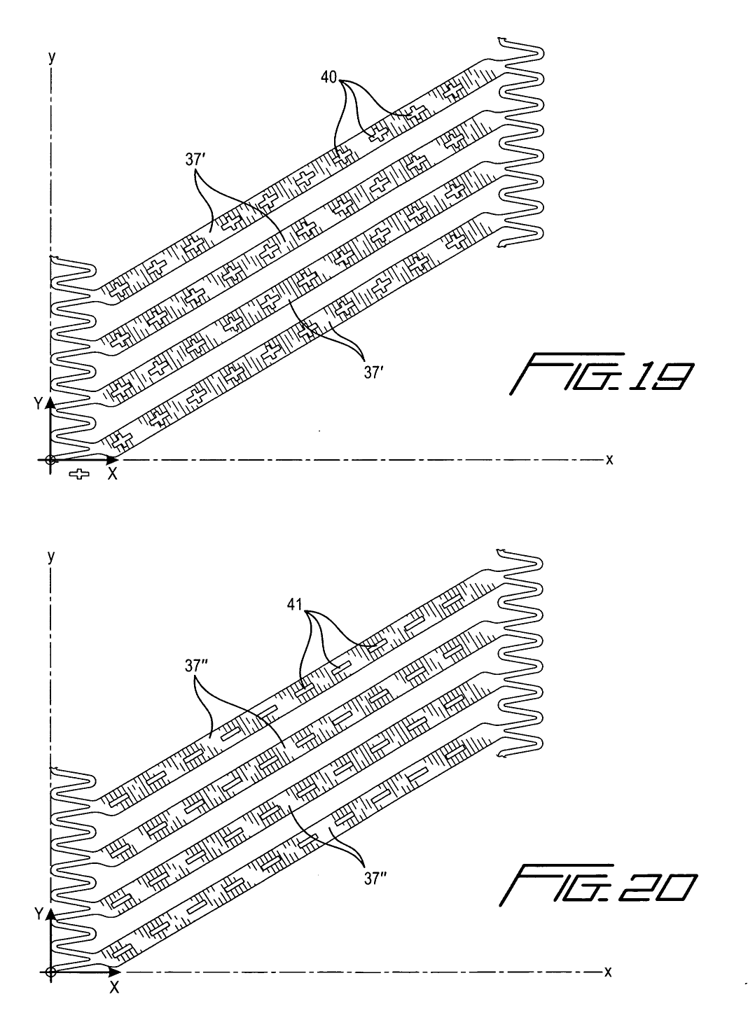

A fourth embodiment is indicated generally at 35 in FIGS. 16-18, wherein the stent body 36 is formed of a plurality of spirally wound, slightly spaced apart bands 37. The bands induce a swirling motion to blood flowing through the stent, thereby preventing stagnation of the blood. Further, the inlet end 38 of the stent is slightly outwardly flared as indicated in FIG. 17 to smooth the flow of blood entering the stent and prevent turbulence and shear at this point, aiding in the initiation or transition to a swirling motion in the flow of blood entering the stent.

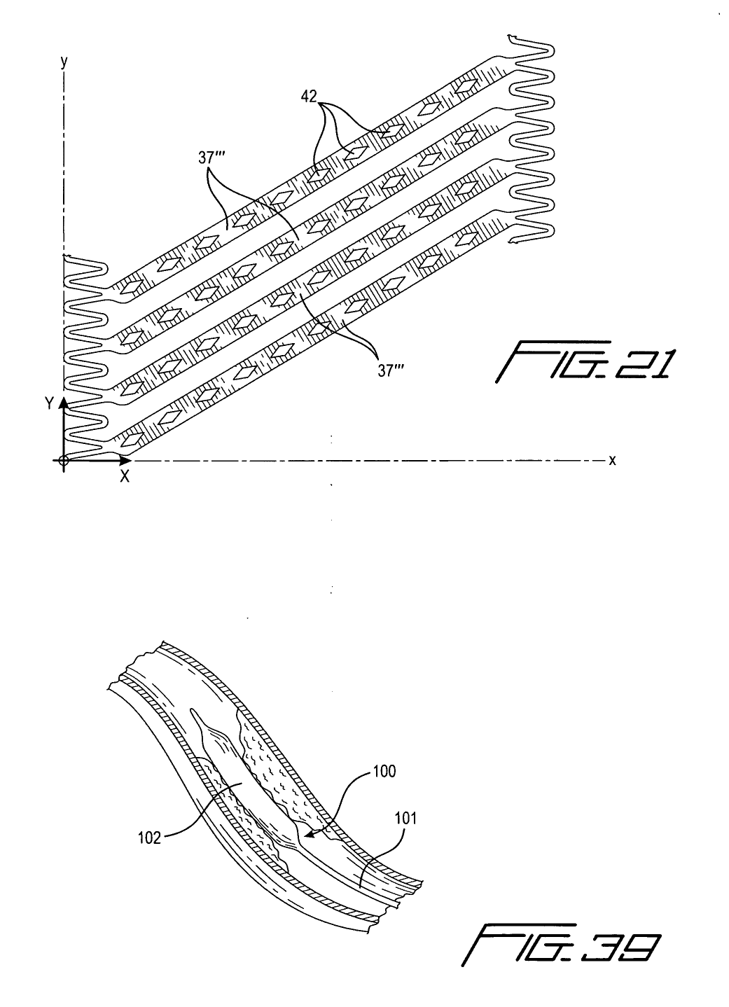

FIGS. 19-21 show variations 37′, 37″, 37′″ of the bands forming the stent in FIG. 16, wherein the surface of the bands is treated as by forming shaped depressions or holes 40, 41 and 42 therein, respectively, to hold a drug or drugs to be carried by the bands. While only depressions or holes are shown, it should be understood that other surface treatments as known in the art could equally as well be used, such as roughening the surface, or first coating it with a polymer, etc.

A fifth embodiment of the first aspect of the invention is indicated generally at 50 in FIGS. 22 and 23. In this form of the invention, the stent body 51 is formed of stacked rings 52, 53, 54 . . . , secured together as by welding or the like to form a hollow tubular structure. The rings preferably comprise dissimilar materials, such as alternating rings of copper, steel and silver. It should be understood that any desired and suitable material could be used for the rings.

A sixth embodiment is indicated generally at 60 in FIG. 24. In this form, the stent body 61 is formed of laminated concentric tubes 62, 63 and 64 each made up of strips or panels 65, 66, 67 of dissimilar materials secured to each other along longitudinal edges and extending the length of the stent body. As shown the strips or panels extend axially of the stent, but they could extend in a spiral or other shape, if desired (not shown). The material of the inner and outer layers or laminations 62 and 64 can be selected for any therapeutic property they may have (e.g., copper, gold, silver, etc.), and the intermediate layer can be selected for strength (e.g., steel, chrome, etc.).

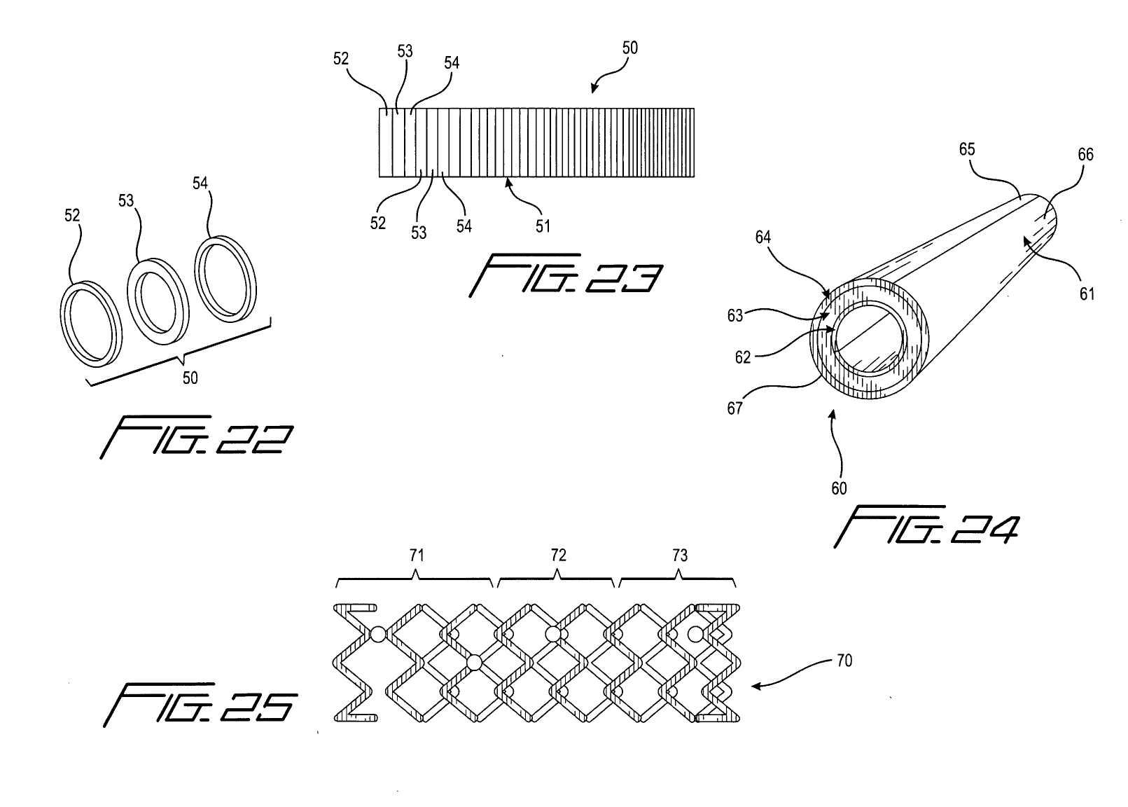

A seventh embodiment is indicated generally at 70 in FIG. 25, wherein different axial segments 71, 72 and 73 of the stent body (shown here as an open lattice design) are formed of different materials. In the specific example shown, one end segment 71 is made of a silver alloy, the center segment 72 is made of a zinc alloy, and the second end segment 73 is made of a copper alloy. The different materials are selected for their different properties.

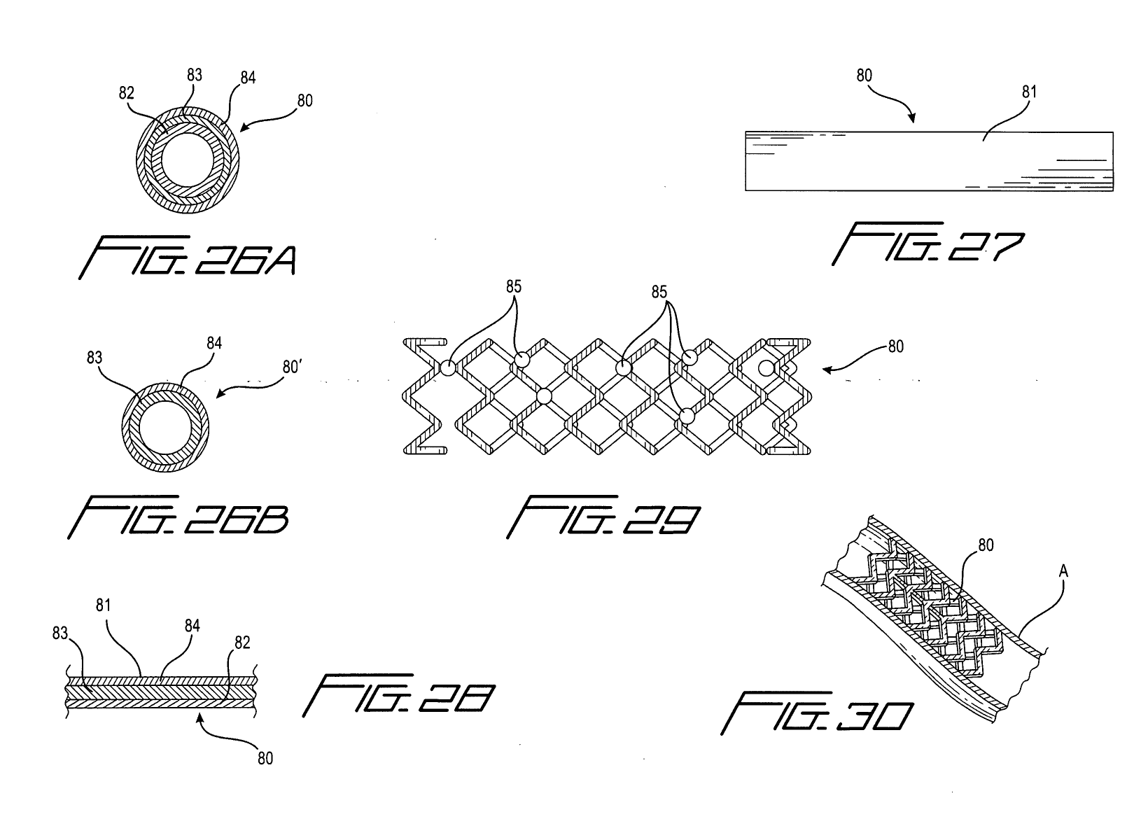

An eighth embodiment is indicated generally at 80 in FIGS. 26A, 27, 28 and 29. In this embodiment the tubular stent body 81 is formed of laminated together concentric tubes 82, 83, 84 of different materials, as in the FIG. 24 embodiment, but the concentric tubes each comprise a single material rather than the panels or strips of the earlier embodiment. FIG. 26B shows an alternate form 80′ wherein only two layers 83 and 84 are used to form the tubular structure. In these forms of the invention, the same material would be exposed along the circumference and length of the stent, but different materials would be exposed at the inner and outer surfaces. FIG. 27 shows the tubular stent body before it is cut to form the open lattice-like structure (see FIG. 29, for example). As shown in FIG. 29, enlarged pads or depots 85 are formed on the stent at selected intersections of the strut elements to carry a drug or drugs. FIG. 30 depicts the stent 80 in place in an artery A.

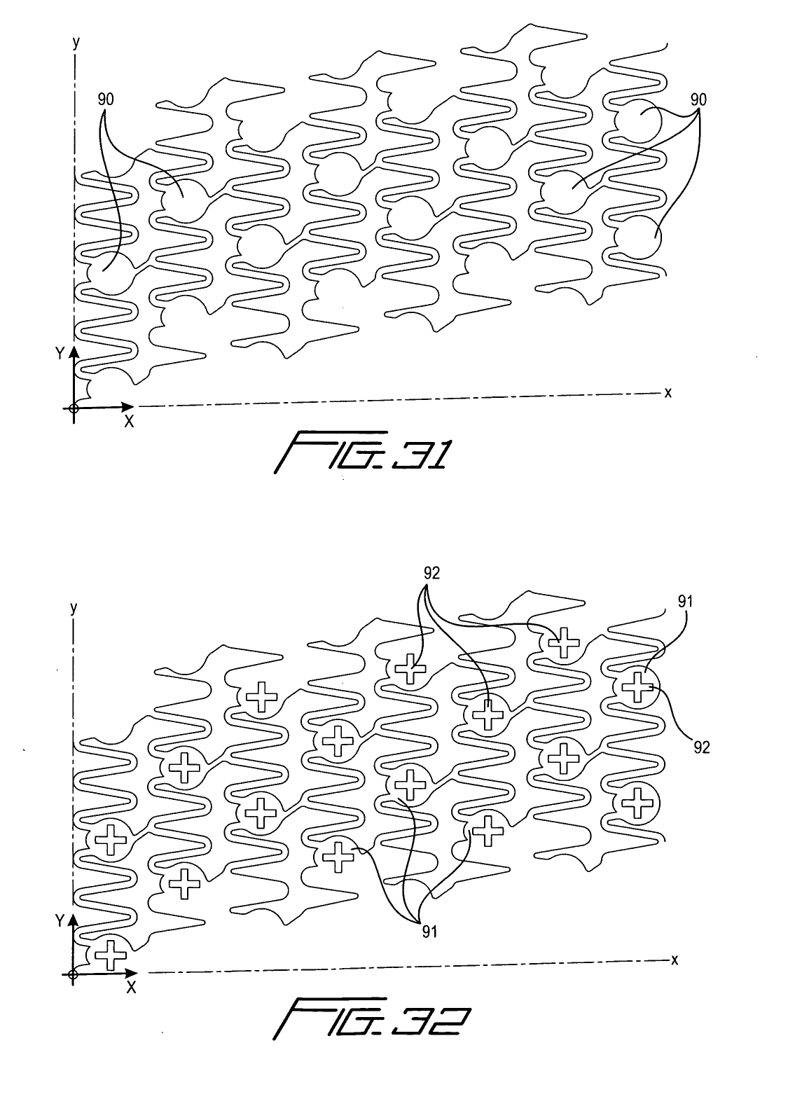

FIGS. 31 and 32 show variations of a stent such as those shown in FIGS. 25 and 29, wherein enlarged pads or depots 90 (FIG. 31) or 91 (FIG. 32) are provided at intersections of the strut elements to carry a drug or drugs. The pads 90 are shown smooth, while the pads 91 are shown with a depression or hole 92 formed thereon to help hold the drug to the pad.

A second aspect of the invention is indicated generally at 100 in FIGS. 33 and 39, which show a catheter 101 having a “repair” device 102 on its distal end for placement at a stenosed site in a body lumen and designed to be left in place for a limited time to treat the stenosis (or restenosis) and then removed. The device is left in place a predetermined time, e.g., 5 to 30 minutes, for appropriate treatment of the site, and is then collapsed and withdrawn from the lumen. For example, the device could be temporarily positioned at a diseased site to dissolve plaque or perform other treatment without the need for implantation of a stent. Or if restenosis occurs in a previously stented site, the device could be placed temporarily at the site to treat the restenosis without the need for implanting a second stent at the site. The device preferably delivers an appropriate therapeutic agent or agents selected for treatment of the diseased site, such as dissolving plaque at the site, or performing other treatment as desired or necessary.

The device is designed so that blood can continue to flow through it while it is in place. FIG. 38 depicts such a structure 103, and another example of such a structure is that described in applicant's copending U.S. patent application Ser. No. 11/252,182, filed Oct. 17, 2005, incorporated by reference herein. In that application, the structure comprises an outer, expandable, open-ended, double-walled cylinder 83 of stretchable elastomeric material and having an inner, cylindrical wall 84 and an outer cylindrical wall 85, defining an annular space 86 therebetween. The space is connected to an inflation tube (not shown) in the catheter so that air or other fluid can be pumped into the space to inflate the cylinder. The cylinder remains collapsed on the distal end of the catheter 81 until the cylinder is positioned at the desired site, whereupon it can be inflated and expanded, with the drug-carrying outer surface of the cylinder pressed against the lumen wall. The space 86 is connected to the inflation tube in the catheter via one or more radially extending members 87. The member 87 preferably is narrow in a direction transverse to the direction of blood flow, whereby it minimally interferes with flow. It can be one or more simple cylindrical tubes (not shown), or an axially elongate structure, or any other suitable connection. Any of these arrangements provide a flow passage through the center of the structure 82 for continuous flow of blood while the device is in place. Moreover, the single radial member shown induces minimal turbulence in blood flowing through the device, but is sufficient to inflate it. Although the device 82 is described as inflatable, it should be understood that other expandable and retractable means could be employed, so long as space is left through the device for continued flow of blood while the device is in place. For instance, a mechanism similar to that used on an umbrella could be employed, with suitable cables or wires extended through the catheter for manipulating linkages to expand and contract the device.

An embodiment of the device is illustrated in FIG. 34, wherein the device comprises a sponge-like structure 105 that can be loaded with a drug or drugs for appropriate treatment of the stenosis. In this form, a catheter would be used to position the sponge at the stenosed site, and the sponge would then be pressed against the stenosis to dissolve it, for example.

An alternate embodiment is shown in FIG. 35, wherein the device comprises an inflatable balloon 110. The outer surface of the balloon could be covered with a drug or drugs that would be pressed against the stenosis to dissolve it, for example.

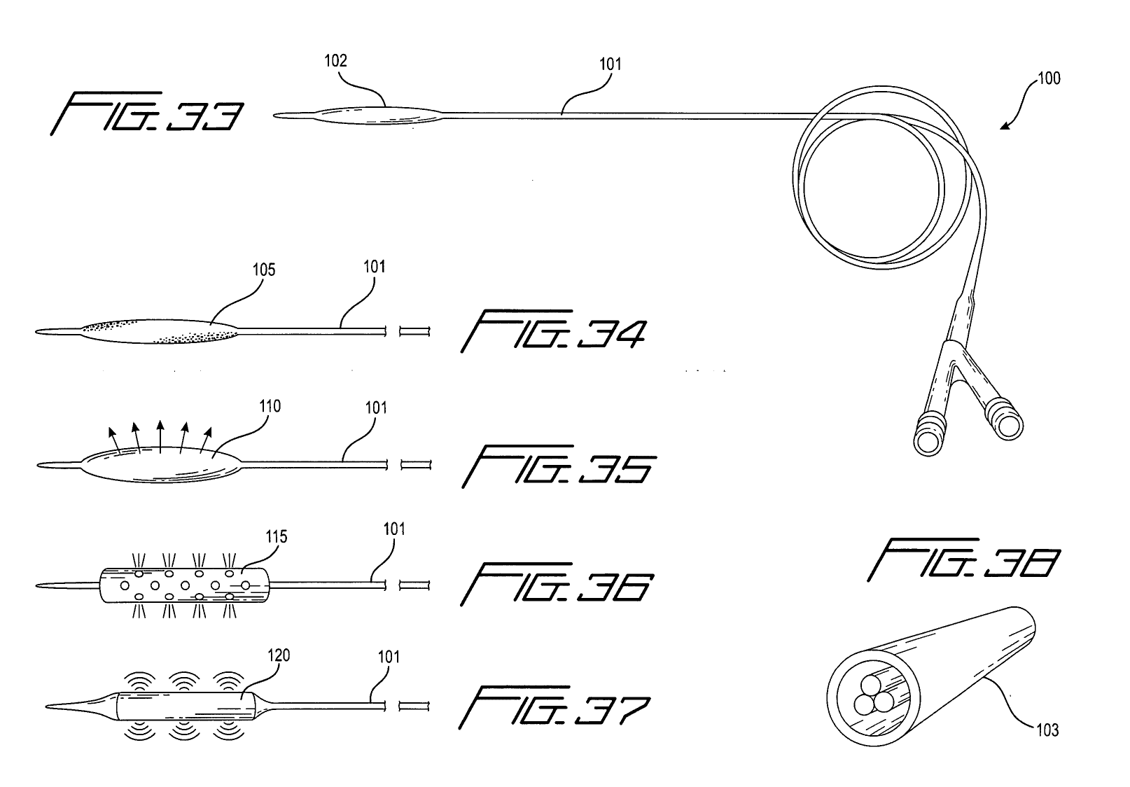

A further embodiment is illustrated in FIG. 36, wherein the device 115 is designed to direct a spray or jets of fluid against the stenosed site. The fluid may carry a drug or drugs if desired. The spray may comprise intermittent jets or pulses of fluid under a moderate pressure, or it may comprise a continuous low pressure flow of a small amount of the treatment agent. Nitrous oxide, for example, could be pumped through the catheter to the site of restenosis to dissolve the restenosis. Further, oxygen could be pumped to the site in lieu of the nitrous oxide, or in combination with the nitrous oxide. For example, nitrous oxide could be pumped to the site for one or two minutes, followed by pumping oxygen to the site for a like period, and then repeating the sequence for a desired time. This procedure could be used, for example, as the primary treatment for stent “repair”.

In the embodiment of FIG. 37, the device 120 uses ultrasound to treat the stenosis.

FIG. 39 shows a catheter-mounted device according to any of the preceding embodiments in place at a stenosed site.

While particular embodiments of the invention have been illustrated and described in detail herein, it should be understood that various changes and modifications may be made in the invention without departing from the spirit and intent of the invention as defined by the appended claims.

What is claimed is:

1. A stent for implantation into a treatment site in a body lumen, comprising:
an elongate, open-ended tubular stent body having a sidewall of interconnected lattice elements or struts defining a plurality of openings through the sidewall, said sidewall being movable from a collapsed position on an end of a catheter for insertion into a body lumen, to a radially expanded position engaged against an inner surface of the body lumen; and
a separate cover carried on an outer surface of the tubular stent body, covering said plurality of openings and being expandable and contractible with the stent body and having an outer surface for carrying at least one therapeutic agent.

2. A stent as claimed in claim 1, wherein:
said cover comprises a longitudinally pleated structure.

3. A stent as claimed in claim 1, wherein:
said cover comprises a member coiled around the stent body.

4. A stent as claimed in claim 1, wherein:
said cover comprises a plurality of overlapping plates.

5. A stent as claimed in claim 3, wherein:
said cover comprises a plurality of bands spirally wound around the stent body.

6. A stent for implantation into a treatment site in a body lumen, comprising:
an elongate, open-ended tubular stent body formed of closely spaced spirally wound bands that impart a swirling motion to blood flowing through the stent.

7. A stent as claimed in claim 6, wherein:
an inlet end of the stent body is slightly outwardly flared to smooth flow of blood entering the stent.

8. A stent for implantation into a treatment site in a body lumen, wherein:
the stent is made of different materials at different portions thereof so that different materials are exposed to body tissue at different locations on the stent.

9. A stent as claimed in claim 8, wherein:
a plurality of rings of dissimilar materials are stacked and secured together to form a tubular stent body that exposes different materials to body tissue at different places along the length of the stent.

10. A stent as claimed in claim 8, wherein:
the stent body comprises concentric layers or tubes of different materials laminated together.

11. A stent as claimed in claim 10, wherein:
at least one of the layers is formed of strips or panels of different materials arranged side-by-side and extending the length of the stent.

12. A stent as claimed in claim 8, wherein:
said stent body comprises a plurality of strut elements connected to form an open lattice-like structure, said lattice-like structure being formed of different materials in different sections along its length.

13. A device for temporary implantation at a treatment site in a body lumen to treat the site, wherein:
said device is attached to a distal end of a catheter for insertion into a body lumen and removal from the lumen, said device being movable between a collapsed position on the distal end of the catheter for insertion into and removal from the body lumen, and a radially outwardly expanded position engaged against an inner surface of the body lumen during a treatment procedure, said device and catheter being left in place in the body lumen during the procedure.

14. A device as claimed in claim 13, wherein:
a therapeutic agent is carried on an outer surface of said device.

15. A device as claimed in claim 13, wherein:
said device has a central, longitudinally extending opening through which fluid can continue to flow while the device is in an expanded position in the body lumen.

16. A device as claimed in claim 15, wherein:
said device is made of a stretchable elastomeric material, and is inflated to move it from its collapsed position to its expanded position, and deflated to move it from its expanded position to its collapsed position.

17. A device as claimed in claim 14, wherein:
said therapeutic agent comprises a plaque-dissolving agent.

18. A process for treating a stenosed site in a body lumen, comprising the steps of:
providing a device on a distal end of a catheter for insertion into a body lumen;
providing a therapeutic agent on the device for treating the stenosed site;
inserting the catheter and device into a body lumen and positioning the device at the stenosed site;
leaving the catheter and device in place in the body lumen for a predetermined limited time to permit the therapeutic agent to act on the stenosed site; and
removing the catheter and device from the body lumen.

19. A medical device for insertion into a stenosed site in a body lumen to treat and remove the stenosis, comprising:
an elongate tubular body having an open inlet end, an open outlet end, and a sidewall, said body being movable from a collapsed position on an end of a catheter for insertion into a body lumen, to a radially expanded position engaged against an inner surface of the body lumen, wherein said open inlet end is outwardly flared to provide smooth entry for fluid flowing through said body, thereby reducing turbulence in said fluid.

20. A medical device as claimed in claim 19, wherein:
a gel-like substance is coated on at least an outer surface of said body, said gel-like substance being selected from the group consisting of a cholesterol-dissolving agent and a blood clot dissolving agent.
